(19) United States
(12) Patent Application Publication
(10) Pub. No.: US 2009/0017107 A1
(43) Pub. Date: Jan. 15, 2009

(54) NOVEL COMPOSITION TO INCREASE LIBIDO

(75) Inventor: Michael Farber, Montreal (CA)

Correspondence Address: BERESKIN AND PARR, 40 KING STREET WEST, BOX 401, TORONTO, ON, M5H 3Y2, CA

(73) Assignee: ORAL DELIVERY TECHNOLOGY LTD., Montreal (CA)

(21) Appl. No.: 12/172,578

(22) Filed: Jul. 14, 2008

Related U.S. Application Data
(60) Provisional application No. 60/949,631, filed on Jul. 13, 2007.

(51) Int. Cl. A61K 31/568; A61K 9/127; A61P 15/00

(52) U.S. Cl. 424/450; 514/170

(57) ABSTRACT

The present invention relates to a composition for increasing testosterone physiological levels comprising a sufficient amount of at least two ketosteroid derivatives of testosterone metabolism in association with a liposomal carrier bound to a saliva-absorbing carrier, wherein said increase in testosterone levels increases libido.

### BACKGROUND OF THE INVENTION

(a) Field of the Invention

In accordance with the present invention, there is provided a composition for increasing testosterone physiological levels comprising a sufficient amount of at least two ketosteroid derivatives of testosterone metabolism in association with a liposomal carrier bound to a saliva-absorbing carrier, wherein said increase in testosterone levels increases libido.

(b) Description of Prior Art

Testosterone is the principal male androgen and is responsible for the development and maintenance of male sexual characteristics, including external virilization, sexual maturity at puberty, spermatogenesis, sexual behavior/libido and erectile functioning. It also supports bone and muscle tissue development during growth. However, after physical maturity, the level of circulating testosterone starts to decline, possibly leading to a diminution in muscle mass. Therefore, there is a growing need for the development of some form of androgen replacement, for the treatment of the various side effects associated with this condition, such as the decrease in libido, sexual functioning and overall sense of being later in life.

Several methods for re-establishing androgens levels to their pre-adult concentrations in men were developed with injectable preparations. U.S. Pat. No. 6,989,378, between others, relates to a novel androgen, (7α,17β)-7-methyl-17-[(1-ox-oundecyl)oxy]estr-4-en-3one (MENT undecanoate), having a good solubility in oily media and being particularly suitable for administration by means of injection. Injectable medias are normally fashioned in order to allow slow and sustained hormone release in the blood of the patient over various preset periods of time. However, the main problem with such inventions is that it usually end-up providing inconsistent dosing because of a great variance in hormone release between the site of injection and the rest of the body. Moreover, injection of testosterone preparations usually entails very high concentrations from the moment of the administration followed by a period of subnormal hormone concentrations. Because of such uncontrolled release of the active agent, various side effects developed in periods of high hormone concentrations, such as gynecomastia, water retention, edema and increased fat deposition.

Some methods of treatment for restoring the testosterone concentrations in adult males with declining levels focused on the administration of a metabolic precursor of testosterone. U.S. Pat. No. 5,880,117 relates to a method of administering the testosterone precursor 4-androstene-3,17β-diol as a means of increasing testosterone levels in humans. The invention proposes a compound which concentration peaks within 90 minutes of its administration and declines thereafter over a period of 3 to 4 hours. Even if the androgen preparation has shown easy conversion to testosterone in the physiologic environment, it still lacks constant repartition in the organs of predilection and can possibly entail various side effects. U.S. Pat. No. 6,451,782 is based on the administration of 4-androstene-3α,17β-diol, a direct precursor hormone to testosterone, in order to increase testosterone levels in male subjects. However, even if conversion to testosterone has been demonstrated as being much more complete than in other cases, the release kinetic of the compound were still not ideal.

Other proposed methods of treating the present condition were related to the physiologic administration of synthetic androgen derivatives of testosterone such as methyltestosterone, fluoxymesterone and stanozol. Those compounds were alkylated at the 17^{th} carbon in order to restrain any reduction of the metabolite to its inactive from. Such innovation induced an increase in the bioavailability of the compound, which allowed a more constant release of the active agent in the physiologic environment. However, patients encountered possible risks of developing complications with liver functions, which highly diminishes the convenience of using such technology.

Steroidal based aromatase inhibitors, Androsta-1,4,6-triene-3,17-dione (ATD) specifically, have been cited in the literature on numerous occasions over the past thirty years. It was first used to elucidate the nature of the enzyme aromatizing androstenedione and testosterone. The effects of aromatase inhibition upon sex steroids in men (in this case older eugonadal men) were definitively and quantatively studied wherein it was shown that administration of an aromatase inhibitor to 15 men over 65 caused significant decreases in estrogen and its related compounds and significantly increased testosterone.

In order to increase sex drive or libido, the most important things that are necessary are:

1. Blood Circulation

2. Nitric Oxide

3. Testosterone

4. Energy

Blood circulation is critical for any man to perform better and have better erections. Better blood flow to the genitals is a must for erections and also it is the main source of increasing nitric oxide. The main role of nitric oxide is to relax the blood vessels so that they are wide enough to allow more blood into the penis. If one does not have enough of blood circulation, it is not likely to get an erection. Testosterone is the main hormone which is responsible for maintaining sexual function in men. Low testosterone means low sex drive and even lead to erectile or sexual dysfunction among other health deficiencies.

It would therefore be highly desirable to be provided with a potent fast acting aromatase inhibitor displaying only slight or negligible binding to the peripheral androgen receptors that would rapidly raise testosterone levels in male subjects, have a half life allowing for at most twice daily ingestion and have sufficient binding to the hypothalamic androgen receptor sites to down-regulate the feedback loop.

### SUMMARY OF THE INVENTION

In accordance with a preferred embodiment of the present invention, there is provided a composition for increasing testosterone physiological levels comprising a sufficient amount of at least two ketosteroid derivatives of testosterone metabolism in association with a liposomal carrier bound to a saliva-absorbing carrier, wherein said increase in testosterone levels increases libido.

In accordance with another preferred embodiment of the present invention, the composition comprises four ketosteroid derivatives of testosterone metabolism.

In accordance with another preferred embodiment of the present invention, the ketosteroid derivatives of testosterone are chosen from:
3,17-diketoandrost-1,4,6-triene
6-methylene-3-keto-17-hydroxylandrost-1,4-diene
6-bromo-α-3,17-diketoandrost-1,4-diene; and
6-bromo-β-3,17-diketoandrost-1,4-diene.

In accordance with another preferred embodiment of the present invention, the four ketosteroid derivatives of testosterone comprised in the composition are:
3,17-diketoandrost-1,4,6-triene
6-methylene-3-keto-17-hydroxylandrost-1,4-diene
6-bromo-α-3,17-diketoandrost-1,4-diene; and
6-bromo-β-3,17-diketoandrost-1,4-diene.

In accordance with another preferred embodiment of the present invention, the saliva-absorbing carrier is microcrystalline cellulose.

In accordance with another preferred embodiment of the present invention, the composition of the present invention further comprises an aphrodisiac compound.

In accordance with another preferred embodiment of the present invention, the aphrodisiac compound is a tropane alkaloid, Yohombine, Bremelanotide and phenylethylamine (PEA).

In accordance with another preferred embodiment of the present invention, the ketosteroid derivatives of testosterone metabolism are natural or synthetic.

In accordance with another preferred embodiment of the present invention, the testosterone levels are increased to supraphysiological levels.

In accordance with another preferred embodiment of present invention, there is provided a method for increasing testosterone physiological levels in a male subject, which comprises administering a sufficient amount of the composition of the present invention.

In accordance with another preferred embodiment of the present invention, administration of the composition is peroral, transdermal or intranasal.

In accordance with another preferred embodiment of the present invention, administration of the composition is sublingual.

In accordance with another preferred embodiment of the present invention, the daily total dosage of the composition to administer is of about 25 to 1000 mg.

In accordance with another preferred embodiment of the present invention, the daily total dosage is administered at least two times a day.

In accordance with another preferred embodiment of the present invention, the daily total dosage is divided in two equal dosages to be administered at twelve hours intervals.

In accordance with another preferred embodiment of the present invention, the daily total dosage is divided in three equal dosages to be administered at eight hours intervals.

In accordance with another preferred embodiment of the present invention, the testosterone level is significantly increased.

In accordance with another preferred embodiment of the present invention, there is provided a method for improving a male's libido, which comprises administering a sufficient amount of the composition of the present invention.

In accordance with another preferred embodiment of the present invention, the administration of the present composition is peroral, transdermal or intranasal.

In accordance with another preferred embodiment of the present invention, the administration is sublingual.

In accordance with another preferred embodiment of the present invention, the daily total dosage of the composition to administer is of about 25 to 1000 mg.

In accordance with another preferred embodiment of the present invention, the daily total dosage is administered once a day within one hour of the sexual activity.

All references referred herein are hereby incorporated by reference.

For the purpose of the present invention, the following terms are defined below.

The expression “supraphysiological levels” as used herein refers to amounts greater than normally found in the human body.

The expression “significantly increased” refers to a rapid and higher production than normal physiological levels in the human body.

### BRIEF DESCRIPTION OF THE DRAWINGS

FIG. 1 illustrates the molecular structure of 3,17-diketoandrost-1,4,6-triene compound.

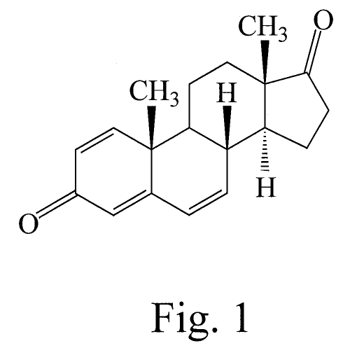

FIG. 2 illustrates the molecular structure of the 6-methylene-3-ketoandrost-1,4-diene-17-ol.

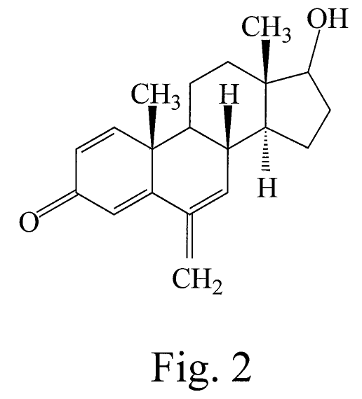

FIG. 3 illustrates a graph of testosterone levels of patients after taking a composition of the present invention.

### DETAILED DESCRIPTION OF THE INVENTION

In accordance with the present invention, there is provided a composition for increasing testosterone physiological levels comprising a sufficient amount of at least two ketosteroid derivatives of testosterone metabolism in association with a liposomal carrier bound to a saliva-absorbing carrier, wherein said increase in testosterone levels increases libido.

The increase in testosterone levels in the present invention is generated through administration of preparations of testosterone ketostreroid metabolic derivatives. Those derivatives are the products of the ketosteroid reduction of this androgen by members of an enzymatic family present in high quantities in steroid target tissues and catalyzing many key reactions: the 3α-hydroxysteroid dehydrogenase family. These enzymes are at the center of various reactions of activation and deactivation of male and female sex hormones, therefore protecting the tissue against circulating hormone excess. The enzymes that are involved in those reactions are the members of the aldo-keto reductase family, or human 3α-hydroxysteroid dehydrogenase isoforms. In humans, this family holds four different 3α-HSD isoforms: type 1 3α-HSD (AKR1C4) [17,1], type 2 3α(17β)-HSD (AKR1C3), type 3 3α-HSD (AKR1C2) and 20α(3α)-HSD (AKR1C1). Type 2 3α-hydroxysteroid dehydrogenase (3α-HSD) is a multi-functional enzyme that possesses 3α-, 17β- and 20α-HSD, as well as prostaglandin (PG) F synthase activities and catalyzes androgen, estrogen, progestin and PG metabolism. Type 2 3α-HSD was cloned from human prostate, is a member of the aldo-keto reductase (AKR) superfamily and was named AKR1C3. In androgen target tissues such as the prostate, AKR1C3 catalyzes the conversion of Δ(4)-androstene-3,17-dione to the active testosterone metabolite, testosterone to its inactive C-17-ketosteroids reduced form, the very potent 5α-dihydrotestosterone to the 5α-androstane-3α,17β-diol (3α-diol) inactive metabolite, and 3α-diol to androsterone. Thus, AKR1C3 regulates the balance of androgens and hence trans-activation of the androgen receptor in the prostate, by modulating the concentration of circulating steroid hormone, and therefore generate the growth of the gland and related muscle size and strength increase. Indeed, tissue distribution studies indicate that AKR1C3 transcripts are highly expressed in human prostate.

The main mechanism of action utilized through this invention consists in competitively inhibiting the metabolism of the AKR1C3 isoform of the 3α-hydroxysteroid dehydrogenase family. Indeed, since the metabolic reactions catalyzed by this enzyme is reversible, by introducing testosterone AKR1C3 metabolic derivative, which is 3,17-diketoandrost-1,4,6-triene, the enzyme will bind to the newly introduced substrate whether than to the circulating testosterone. Thus, whether than degrading testosterone, AKR1C3 will actually perform an oxidative metabolism on the presented 3,17-diketoandrost-1,4,6-triene molecule that will result in the production of testosterone. Therefore, by administrating testosterone metabolic derivatives in the milieu, the enzyme is inhibited in its ketosteroid reduction of testosterone in inactive metabolites, and will instead perform the inverse reaction, consisting in transforming testosterone inactive metabolites in testosterone potent molecules. Thus, testosterone levels are increased by metabolism of the androgen from its inactive metabolite and related inhibition of the metabolism of the physiologic circulating hormone.

The composition has sufficient amounts of ATD to interfere with the circulating aromatase enzymes over a period of twelve hours after initial inhibition by the 6 bromo-α-3,27-diketoandrost-1,4-diene and 6 bromo-β-3,27-diketoandrost-1,4-diene compounds but not enough ATD to have antagonistic competition for the peripheral androgens. Therefore, the ratios of the various compounds in the composition are determined by the need for rapid and effective downregulation of the aromatase enzymes by the 6 bromo-α-3,27-diketoandrost-1,4-diene and 6 bromo-β-3,27-diketoandrost-1,4-diene compounds but only maintenance of that state by ADT with the added 6-methylene-3-keto-17-hydroxylandrost-1,4-diene compound so that effective binding of the hypothalamic receptor occurs to interfere with the downregulation of testosterone at the hypothalamus.

The liposomal carrier of the composition allows faster uptake, no degradation at the intestinal uptake and liver and also no variations among individuals due to transit time, degradation of capsules and the like, so that timing for use can be precise.

The present invention will be more readily understood by referring to the following examples which are given to illustrate the invention rather than to limit its scope.

### EXAMPLE 1

### Evaluation of Free Testosterone Levels in Subjects Before and after Administration of the Composition

Material: The composition for oral administration contains a complex of 3,17-diketoandrost-1,4,6-triene and the 6-bromo-androst-1,4-diene-3,17dione-α/β isomers. The molecular structure of these compounds are provided in Formula 1 to 3 below.

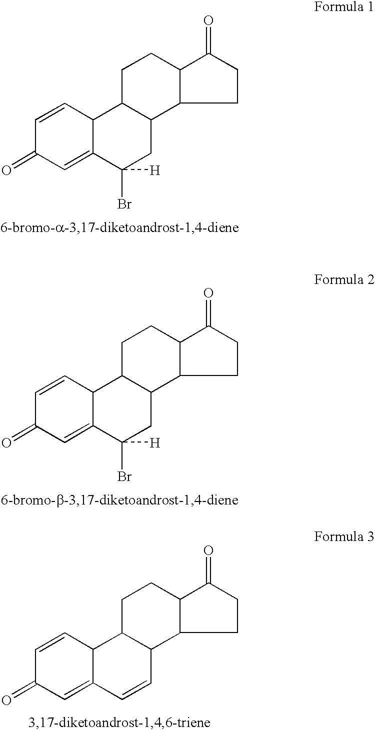

Method: The composition was tested in laboratory to validate its free testosterone boosting effects in a doctor controlled medical trial. The test results revealed that male subjects showed free testosterone levels that were increased up to 10,000% above baseline levels within 1 hour after taking the composition. After 2 hours, testosterone levels remained elevated up to 8.536% above pre-treatment baseline levels. These supraphysiological levels of free testosterone allow for maximum tissue saturation and powerful anabolic effects. It has been demonstrated that there is a strong correlation between steroid hormone levels in saliva and the amount of hormone in the blood that is active or “bioavailable.” It is this fraction of total hormone that is free to enter the target tissues like the muscles. These tests confirm that the composition is extremely effective in raising free testosterone.

Results: The results are provided in TABLE 2 and TABLE 3.

TABLE 2

| | | Free Testosterone | Free Testosterone |
|---|---|---|---|
| Male | Free | 1 hour after | 2 hours After |
| Subjects | Testosterone | administration of | administration of |
| Ages | Befor ve | composition | composition |
| 24 to 50 | administration | picograms/ml and | picograms/ml and |
| years | of composition | percentage increase | percentage increase |
| Subject 1 | 89 pg/ml | 5430 pg/ml | 2076 pg/ml |
| | | (+6101%) | (+2333%) |
| Subject 2 | 54 pg/ml | 5520 pg/ml | 5320 pg/ml |
| | | (+10222%) | (+9852%) |
| Subject 3 | 66 pg/ml | 5727 pg/ml | 5634 pg/ml |
| | | (+8677%) | (+8536%) |
| Subject 4 | 53 pg/ml | 6439 pg/ml | 4874 pg/ml |
| | | (+12149%) | (+9196%) |
| Average of 4 | 65.5 pg/ml | 5779 pg/ml | 4476 pg/ml |
| Subjects | | (+8823%) | (+6834%) |

TABLE 3

| | Average Free Testosterone Levels of Test |
|---|---|
| Average Free Testosterone | Subjects following administration of |
| Levels of Test | composition |
| Subjects | 60 min and 120 min |
| 65.5 picograms | 5779 picograms per milliliter at 60 min |
| | 4476 picograms per milliliter at 120 min |

*Note:
Normal Healthy Adult Males Testosterone Ranges are 44-148 picograms per milliliter.

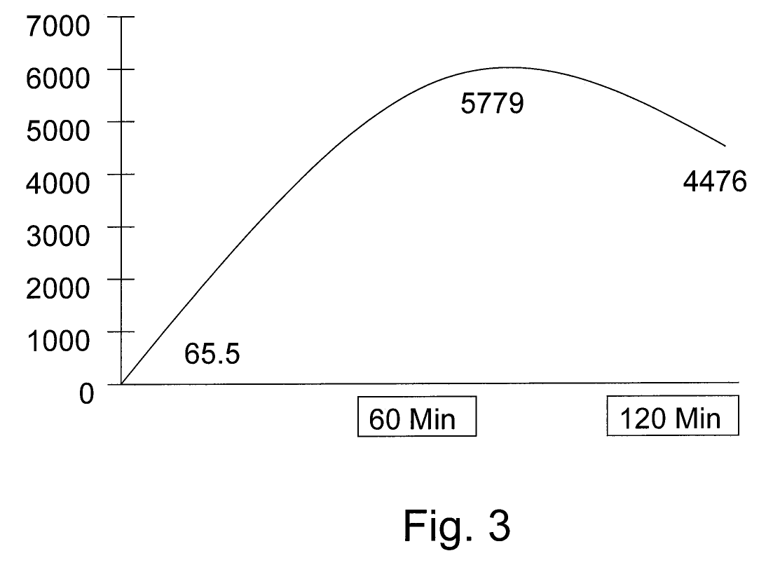

The average free testosterone values of the test subjects were plotted to show the dramatic rise in free testosterone levels and the sustained effect over time. Even after 120 minutes, free testosterone levels were still greatly elevated above baseline levels of the subjects and normal range levels. These results are summarized in FIG. 3.

As a result of this researched-based product development effort, the composition of the present invention features a unique aromatase-inhibitor complex bio-engineered to produce a precise succession of testosterone elevating action via fast acting aromatase-inhibiting ingredients.

Sublingual delivery is a method of systemic nutrient delivery that offers several advantages, as the oral mucosa is highly vascularised. Certain nutrients that are absorbed through the oral mucosa directly enter the systemic circulation, bypassing the gastrointestinal tract and first-pass metabolism in the liver.

Clinical evaluation of aging males has shown that increasing levels of endogenous testosterone leads to improved mental function, mood, libido, and with that attainment of high enough levels that even lean muscle mass accretion can be positively affected.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A composition for increasing testosterone physiological levels comprising a sufficient amount of at least two ketosteroid derivatives of testosterone metabolism in association with a liposomal carrier bound to a saliva-absorbing carrier.

2. The composition according to claim 1, wherein said increase in testosterone levels increases libido.

3. The composition according to claim 1, wherein said composition comprises four ketosteroid derivatives of testosterone metabolism.

4. The composition according to claim 1, wherein said ketosteroid derivatives of testosterone are chosen from:
3,17-diketoandrost-1,4,6-triene
6-methylene-3-keto-17-hydroxylandrost-1,4-diene
6-bromo-α-3,17-diketoandrost-1,4-diene; and
6-bromo-β-3,17-diketoandrost-1,4-diene.

5. The composition according to claim 3, wherein said four ketosteroid derivatives of testosterone's metabolism are:
3,17-diketoandrost-1,4,6-triene
6-methylene-3-keto-17-hydroxylandrost-1,4-diene
6-bromo-α-3,17-diketoandrost-1,4-diene; and
6-bromo-β-3,17-diketoandrost-1,4-diene.

6. The composition according to claim 1, wherein said saliva-absorbing carrier is microcrystalline cellulose.

7. The composition according to claim 1, which further comprises an aphrodisiac compound.

8. The composition according to claim 7, wherein said aphrodisiac compound is a tropane alkaloid, Yohombine, Bremelanotide and phenylethylamine (PEA).

9. The composition according to claim 1, wherein said ketosteroid derivatives of testosterone metabolism are natural or synthetic.

10. The method according to claim 1, wherein said testosterone levels are increased to supraphysiological levels.

11. A method for increasing testosterone physiological levels in a male subject, which comprises administering a sufficient amount of the composition of claim 1.

12. The method according to claim 11, wherein said administration is peroral, transdermal or intranasal.

13. The method according to claim 11, wherein said administration is sublingual.

14. The method according to claim 11, wherein said amount is a daily total dosage of about 25 to 1000 mg.

15. The method according to claim 14, wherein said daily total dosage is administered at least two times a day.

16. The method according to claim 15, wherein said daily total dosage is divided in two equal dosages to be administered at twelve hours intervals.

17. The method according to claim 15, wherein said daily total dosage is divided in three equal dosages to be administered at eight hours intervals.

18. The method according to claim 11, wherein the testosterone level is significantly increased.

19. A method for improving a male's libido, which comprises administering a sufficient amount of the composition of claim 1.

20. The method according to claim 19, wherein said administration is peroral, transdermal or intranasal.

21. The method according to claim 19, wherein said administration is sublingual.

22. The method according to claim 19, wherein said amount is a daily total dosage of about 25 to 1000 mg.

23. The method according to claim 22, wherein said daily total dosage is administered once a day within one hour of the sexual activity.
